(19) United States
(12) Patent Application Publication
(10) Pub. No.: US 2011/0111435 A1
(43) Pub. Date: May 12, 2011

(54) Detecting Cell Surface Markers

(75) Inventors: Lynne DOBSON, County Dublin (IE); Donal O'SHEA, County Meath (IE); Catherine CONWAY, Dublin 15 (IE); Alan HANLEY, County Galway (IE)

(73) Assignee: SlidePath Limited, Dublin 9 (IE)

(21) Appl. No.: 12/613,798

(22) Filed: Nov. 6, 2009

(51) Int. Cl. G01N 33/574

(52) U.S. Cl. 435/7.23

(57) ABSTRACT

In one aspect, the present invention provides a method for detecting an expression level of a cell surface marker in a sample, comprising staining the sample with a reagent that labels the cell surface marker; obtaining an image of the stained sample; and determining a value for continuity of cell surface staining in the image, wherein the value is indicative of the expression level.

### FIELD OF THE INVENTION

The present invention relates to the field of detection and quantitation of cell surface markers, for instance in the automated analysis of stained tissue samples for the diagnosis and treatment of disease.

### BACKGROUND OF THE INVENTION

Targeted therapeutics or personalised medicine regimes are driving a new era of integrated diagnostics and therapeutics, particularly in the oncology domain. Many anti-cancer antibodies have been approved in association with companion tests for biomarker expression to identify the most responsive patients, ensuring that accurate evaluation of biomarker status has become particularly acute in the clinical laboratory.

The role that biomarkers can play is exemplified by HER-2: a prognostic, predictive and therapy selection factor for patients with breast cancer. Amplification of the HER-2 gene or over-expression of its protein product in cell membranes is seen in 10-30% of invasive breast cancer and is associated with increased disease recurrence and poor prognosis. Clinically, HER-2 is important as the target of the monoclonal antibody trastuzumab (Herceptin®) which significantly improves response rate, disease progression and overall survival when used in the adjuvant setting compared with chemotherapy alone. The association between HER-2 expression and Herceptin® response has led to the recommendation that this parameter should be evaluated in every primary invasive breast cancer to distinguish those patients for whom the drug may be of benefit, not only because of the expense of treatment, but also because of its potential to cause myocardial toxicity if incorrectly prescribed.

The therapeutic relevance of HER-2 status demands highly reliable and robust testing to identify tumours which over-express this protein. The recommended evaluation method for HER-2 is immunohistochemistry (IHC) to detect expression of the HER-2 protein in cell membranes, with equivocal cases confirmed at the gene expression level using fluorescent in-situ hybridisation (FISH). FISH is considered the gold-standard method of evaluation affording an objective and quantitative scoring system; however this technique suffers from fading fluorochromes and thus poor long term stability, in addition to a requirement for specialised microscopic equipment which restricts its use in conventional laboratories. Chromogenic ISH (CISH) or Silver enhanced ISH (SISH) which do not rely on fluorescent microscopy represent alternatives to FISH in terms of HER-2 oncogene analysis. However, although these methods have been determined to give comparable results to FISH, they are not yet widely utilised in diagnostic pathology.

In contrast, qualitative IHC testing is advocated as the primary assay for identifying trastuzumab candidates because it is readily available, easily performed in most clinical pathology laboratories and has many advantages over FISH or CISH in terms of economics, as well as being highly amenable to automation. Nonetheless, despite efforts to standardise assay protocol and interpretation, antibodies and methods vary across laboratories and IHC scoring remains an inherently subjective process to which only limited statistical confidence can be assigned due to inherent observer variability and the semi-quantitative nature of the data. Even for the trained eye of a pathologist, accurate distinction between the nominal categories (0, 1+, 2+, 3+) is difficult and often arbitrary, and significant variation is introduced as a result of over-using the intermediate category during reviews.

Recently, high rates of discordance between IHC reviewed at high-volume HER-2 reference centres and low-volume regional laboratories has cast doubt on the reliability of results. As this stands alone in determining which patients are likely to respond to trastuzumab therapy, additional attention to the performance and interpretation of IHC testing is now warranted. Participation in external quality assurance (EQA) schemes is recommended and, according to the updated NCCN guidelines, if standards cannot be met material should be sent to a reference laboratory.

Nonetheless, whilst the current EQA schemes assess methodologies they do not attend to disparity in interpretation; in order to address this the American Society of Clinical Oncology has suggested that image analysis could be an effective tool for achieving consistency. Indeed, virtual pathology, the process of assessing digital images of histology slides, is gaining momentum in today's laboratory environment, with digital image acquisition systems and associated image analysis solutions viewed by most as the next critical step. Image analysis may serve to reduce scoring variability by providing a quantitative HER-2 reference tool, thus standardising the evaluation system.

Despite the advances provided by automated image acquisition and analysis systems, it has been found that the results of automated image analysis of IHC-treated samples do not always correlate well with manual review or more accurate methods such as FISH. Accordingly, there is still a need to develop automated methods that can more accurately quantitate biomarkers such as HER-2 in a tissue sample.

### SUMMARY OF THE INVENTION

In one aspect, the present invention provides a method for detecting an expression level of a cell surface marker in a sample, comprising staining the sample with a reagent that labels the cell surface marker; obtaining an image of the stained sample; and determining a value for continuity of cell surface staining in the image, wherein the value is indicative of the expression level.

In one embodiment, the cell surface marker is a membrane protein, such as a growth factor receptor. The cell surface marker may be associated with a disease, such as cancer. Preferably the cell surface marker is HER-2.

In one embodiment, step (a) comprises staining the sample by immunohistochemistry using an antibody specific for the cell surface marker. In another embodiment, steps (b) and (c) are performed by an automated image capture and analysis system.

In another aspect, the present invention provides an automated method for analyzing an image of a stained tissue sample, comprising determining a value for continuity of cell surface staining in the image.

In one embodiment, the sample has been stained with a reagent that labels a cell surface marker, and the value is indicative of an expression level of the cell surface marker in the sample.

In one embodiment, pixels in the image representative of positive staining are detected by applying a colour transformation to the pixels, and applying a threshold value to suppress background. Pixels in the image representative of cell surfaces may be determined, for example by detecting pixels surrounding nuclei stained with a counterstain. In one embodiment, the continuity value comprises a percentage of cell surfaces in the image which are continuously stained.

In one embodiment, the method further comprises determining a value for intensity of cell surface staining in the image. In a further embodiment, the continuity value and intensity value are combined to provide a weighted probability value indicative of a probability of the sample being classified in a predefined staining class. Preferably the sample is classified into a staining class indicative of a level of HER-2 expression in the sample.

In another aspect, the invention provides a method for diagnosing a condition associated with expression of a cell surface marker in a subject, comprising detecting an expression level of the cell surface marker in a sample from the subject by a method as defined in claim 1, wherein an elevated expression level of the cell surface marker in the sample compared to a control sample is indicative of the presence of the condition in the subject.

In one embodiment the condition is cancer. Preferably the cell surface marker is HER-2.

In a further aspect, the invention provides a method for predicting responsiveness to therapy with an anti-HER-2 antibody in a subject, comprising detecting an expression level of HER-2 in a sample from the subject by a method as defined in claim 5, wherein an elevated expression level of HER-2 in the sample compared to a control sample is indicative of responsiveness of the subject to therapy with the anti-HER-2 antibody.

In one embodiment, the expression level of HER-2 is classified as a score of 0, 1+, 2+ or 3+, for example wherein a score of 3+ is indicative of responsiveness of the subject to therapy with the anti-HER-2 antibody. A score of 2+ may be considered to be inconclusive for response to anti-HER-2 treatment, e.g. may suggest further investigations are carried out in order to determine responsiveness. A score of 0 or 1+ may be considered to be negative for responsiveness of the subject to anti-HER-2 therapy.

In a further aspect, the invention provides a computer program, residing on a computer-readable medium, for automated image analysis, comprising machine-readable instructions for performing a method comprising determining a value for continuity of cell surface staining in an image of a stained tissue sample.

In a further aspect, the invention provides an automated imaging apparatus, wherein the apparatus is configured to obtain an image of a stained tissue sample, and determine a value for continuity of cell surface staining in the image, wherein the value is indicative of an expression level of a cell surface marker in the sample.

Embodiments of the present invention typically employ a step of determining a value for continuity of cell surface staining It has been surprisingly found that this continuity value is particularly useful in the analysis of images of tissue samples, since it can be used to accurately quantitate a cell surface marker in the sample.

### BRIEF DESCRIPTION OF THE FIGURES

FIG. 1 shows an image of a stained tissue section from a breast biopsy sample for use in the present method. The section has been stained using IHC with an anti-HER-2 antibody. The non-invasive regions of tumour were annotated online by a Pathologist and excluded from analysis, in accordance with clinical guidelines.

FIG. 2 shows: (A) an unprocessed image of breast tissue which has been immunohistochemically stained with antibodies probing for HER-2 protein expression; and (B) areas detected as positive for continuous membrane staining by image analysis are highlighted.

FIG. 3 images of samples where image analysis and manual review agreed on a classification of 0/1+ but gene amplification was determined to be positive by FISH. Cases were stained using HER-2 antibodies from A: Ventana Pathway, B: Ventana Pathway, C: Ventana Pathway, D: Dako Hercep Test, E: Leica Oracle, F: Leica Oracle.

FIG. 4 shows a receiver-operator curve for the manual and image analysis review of 136 informative cases (a curve reaching the upper left corner implies better performance).

FIGS. 5A-F show images schematically representing different stages of the processing of FIG. 15.

FIG. 6 shows the importance of evaluating circumferential membrane staining which enables differentiation of 1+ and equivocal (2+) cases by image analysis. A: 1+ in-house control tissue (FISH Score 1.18); B: 2+ in-house control tissue (FISH Score 1.97); (i): original image; (ii): regions of issue identified as positively-stained membrane; (iii): regions of positively and continuously stained membrane.

FIG. 7 shows examples of tissue samples classified as 2+ (top row) or 1+ (bottom row) for HER-2, and how these may be discriminated by differences in the continuity of membrane staining

FIG. 8 shows a comparison of membrane continuity and membrane absorbance values for tissue samples determined using the present method, with manual classification into HER-2 scoring categories.

FIG. 9 shows a comparison of membrane continuity and membrane absorbance values for tissue samples with classification into HER-2 scoring categories according to the present method.

FIG. 10 shows a comparison of membrane continuity and membrane absorbance values for tissue samples with experimentally-determined FISH score.

FIG. 11 shows a plot of predicted FISH score, estimated by determining a HER-2 score according to the present image analysis method, against experimentally-determined FISH score.

FIG. 12A schematically illustrates a microscope system for capturing images of a sample.

FIG. 12B schematically illustrates the microscope system of FIG. 12A connected to a server and network.

FIG. 13 schematically illustrates a general purpose computer.

FIG. 14 schematically illustrates a hospital intranet connected to the internet; and

FIG. 15 is a flow diagram schematically representing a method for processing an image according to an embodiment of the invention.

### DETAILED DESCRIPTION OF THE INVENTION

Detecting a Cell Surface Marker

In one embodiment, the present invention relates to a method for detecting an expression level of a cell surface marker in a sample. By “detecting an expression level” it is meant that the amount of the cell surface marker in the sample is measured or quantitated. The cell surface marker may be any molecule of interest which is present on the surface of cells. Typically the “cell surface” comprises the cell (cytoplasmic) membrane, particularly in the case of animal cells, including associated lipids, carbohydrates and proteins and the intracellular and extracellular portions thereof. In other embodiments, the cell surface may additionally comprise one or more further components, such as the cell wall, for instance in the case of plant or bacterial cells, or outer bacterial membrane in the case of gram-negative bacteria.

By “marker” it is meant any biological molecule (or fragment thereof) of interest, e.g. a biomarker which is present on the cell surface. Such markers include, but are not limited to, biomolecules comprising polypeptides, proteins, carbohydrates, lipids, glycoproteins, ribonucleoproteins, lipoproteins, glycolipids and fragments thereof. Typically the marker comprises a membrane protein, including polypeptides, glycoproteins and lipoproteins. Thus the marker may be a transmembrane protein or may be bound to a transmembrane protein or membrane lipid, for example.

For example, the marker may be a cell surface receptor. The receptor may comprise a tyrosine kinase receptor, such as an erythropoietin receptor, an insulin receptor, a hormone receptor or a cytokine receptor. Preferred tyrosine kinases include fibroblast growth factor (FGF) receptors, platelet-derived growth factor (PDGF) receptors, nerve growth Factor (NGF) receptors, brain-derived neurotrophic Factor (BDNF) receptors, and neurotrophin-3 (NT-3) receptors, and neurotrophin-4 (NT-4) receptors. The receptor may comprise a guanylyl cyclase receptor such as GC-A & GC-B, a receptor for atrial-natriuretic peptide (ANP) and other natriuretic peptides or GC-C, a guanylin receptor.

In a preferred embodiment, the receptor is a growth factor receptor, such as a member of the ErbB or epidermal growth factor receptor (EGFR) family, e.g. EGFR (ErbB1), HER2 (ErbB2), HER3 (ErbB3), and HER4 (ErbB4).

In another embodiment, the marker is a G protein-coupled receptor (GPCR), also known as a seven transmembrane receptor or 7TM receptor. For example, the receptor may comprise a muscarinic acetylcholine receptor, an adenosine receptor, an adrenergic receptor, a GABA-B receptor, an angiotensin receptor, a cannabinoid receptor, a cholecystokinin receptor, a dopamine receptor, a glucagon receptor, a histamine receptor, a olfactory receptor, a opioid receptor, a rhodopsin receptor, a secretin receptor, a serotonin receptor or a somatostatin receptor.

The receptor may comprise an ionotropic receptor, for example a nicotinic acetylcholine receptor, a glycine receptor, a GABA-A or GABA-C receptor, a glutamate receptor, an NMDA receptor, an AMPA receptor, a kainate receptor (Glutamate) or a 5-HT3 receptor.

In another embodiment, the cell surface marker comprises a cluster of differentiation antigen, e.g. CD2, CD3, CD4, CD5, CD7, CD8, CD9, CD10, CD11, CD13, CD15, CD16, CD20, CD21, CD22, CD23, CD24, CD25, CD33, CD34, CD36, CD37, CD38, CD41, CD42, CD44, CD45, CD52, CD57, CD60, CD61, CD64, CD71, CD79, CD80, CD95, CD103, CD117, CD122, CD133, CD134, CD138 or CD154.

In one embodiment, the marker is correlated with a disease, preferably a human or animal disease. For example, the marker may be associated with cancer, for example breast or ovarian cancer. Suitable cancer cell markers may include a receptor or CD antigen mentioned above, or further cancer-cell specific markers such as CA-125 (MUC-16) or CA19-9. In a particularly preferred embodiment, the marker is HER-2, also known as HER2/neu, erbB-2, or EGFR2, which is commonly associated with breast cancer.

Preparing a Sample

By “sample” it is meant to refer to any biological sample, including tissue and cellular samples. For instance, the sample may comprise a collection of similar cells obtained from a tissue of a subject or patient. By “subject” or “patient” is meant any single subject for which therapy is desired, including humans, cattle, dogs, guinea pigs, rabbits, chickens, insects. Also intended to be included as a subject are any subjects involved in clinical research trials not showing any clinical sign of disease, or subjects involved in epidemiological studies, or subjects used as controls.

The source of the sample may be solid tissue as from a fresh, frozen and/or preserved organ or tissue sample or biopsy or aspirate; or cells from any time in gestation or development of the subject. The tissue sample may also be primary or cultured cells or cell lines. The tissue sample may contain compounds which are not naturally intermixed with the tissue in nature such as preservatives, anticoagulants, buffers, fixatives, nutrients, antibiotics, or the like.

The sample comprises cells, on the surface of which the marker which it is desired to quantitate is expressed. Thus the cells of interest may be any type of mammalian cell, particularly primate, more particularly human. The cells may have various stages of differentiation, and may be normal, pre-cancerous, or cancerous, may be fresh tissue, dispersed cells, immature cells, including stem cells, cells of intermediate maturity, and fully matured cells. The cells may be derived from various organs and tissues, including hematopoietic cells, muscle cells, fibroblasts, lung cells, liver cells, cardiac cells, neuronal cells, breast cells, prostate cells, bone cells, kidney cells, mucosal cells, epithelial cells, skin cells, endothelial cells, lymph node cells, thymus cells, endometrial cells, ovarian cells, gastrointestinal tract cells and the like.

Preferably the sample is a solid tissue sample (e.g. a biopsy sample) from a subject suspected of suffering from a disease, particularly cancer. Thus the tissue sample may comprise neoplastic tissue. In one embodiment, the sample comprises a tissue section, such as a fresh, frozen or paraffin-embedded tissue section, typically from a suspected diseased tissue or organ. By “section” of a tissue sample is meant a single part or piece of a tissue sample, e.g. a thin slice of tissue or cells cut from a tissue sample. Multiple sections of tissue samples may be taken and subjected to analysis according to the present invention. For example, in one embodiment the method may be performed on a tissue microarray comprising a plurality of samples on a single slide, e.g. as disclosed in US 2003/0215936. Typically the section is suitable for analysis by microscopy, e.g. visible light or fluorescent microscopy. The section may, for example, be placed on a solid support such as a microscope slide.

The sample may be prepared in a wide variety of ways, depending upon the nature of the cells or tissue, convenience, the homogeneity or heterogeneity of the cells, the stability or fragility of the cells, etc. Techniques which may be used to prepare the sample include cytospins, cell pellets, paraffin-embedded sections, or other specimens that have been frozen or formalin-fixed, and the like. Methods for preparing tissue samples for microscopic analysis are well known in the art.

In particular embodiments, tissue sections may be prepared from samples derived from breast, prostate, ovary, colon, lung, endometrium, stomach, salivary gland or pancreas. The tissue sample can be obtained by a variety of procedures including, but not limited to surgical excision, aspiration or biopsy. The tissue may be fresh or frozen. In one embodiment, the tissue sample is fixed and embedded in paraffin or the like.

The tissue sample may be fixed (i.e. preserved) by conventional methodology. One of skill in the art will appreciate that the choice of a fixative is determined by the purpose for which the tissue is to be histologically stained or otherwise analyzed. The length of fixation depends upon the size of the tissue sample and the fixative used. By way of example, neutral buffered formalin or paraformaldehyde may be used to fix a tissue sample.

Generally, the tissue sample is first fixed and is then dehydrated through an ascending series of alcohols, infiltrated and embedded with paraffin or other sectioning media so that the tissue sample may be sectioned. Alternatively, the tissue may be sectioned and then the sections fixed. The tissue sample may be embedded and processed in paraffin by conventional methodology. Once the tissue sample is embedded, the sample may be sectioned by a microtome or the like. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

If paraffin has been used as the embedding material, the tissue sections are generally deparaffinized and rehydrated to water. The tissue sections may be deparaffinized by several conventional standard methodologies. For example, xylenes and a gradually descending series of alcohols may be used. Alternatively, commercially available deparaffinizing non-organic agents may be used. After deparaffinization, the sections mounted on slides may be stained with one or more morphological stains (counterstains) for evaluation, if required. Generally, the section is stained with one or more dyes each of which distinctly stains different cellular components, for example, a xanthine dye, a thiazine dye or methylene blue. Typically the counterstain is a nuclear stain, in order to facilitate the identification and/or counting of individual cells. Staining may be optimized for a given tissue by increasing or decreasing the length of time the slides remain in the dye.

Staining the Sample for the Cell Surface Marker

One embodiment of the present invention comprises a step of staining the sample with a reagent that labels the cell surface marker. By this it is meant that the reagent enables the marker to be detected, for instance by binding to the marker and providing a detectable signal. Typically the reagent used in this step is selective or specific for the marker, in contrast to the morphological stain discussed above, thereby providing a stain which is uniquely indicative of the presence of the marker. Thus “staining” refers to any step which renders the marker detectable, particularly a histological method which renders the marker detectable by microscopic techniques, such as those using visible or fluorescent light. One or more reagents may be used in combination in this step in order to detect the marker, e.g. a first reagent may bind specifically to the marker and a second reagent may bind to the first reagent and provide the detectable signal.

In one embodiment, the method may use immunohistochemistry (IHC) to specifically stain the sample for the marker of interest. IHC may be performed in combination with morphological staining as discussed in the preceding section (either prior to, but preferably thereafter). In IHC, the marker is detected by an antibody which binds specifically to the cell surface marker.

The antibody may be a monoclonal antibody, polyclonal antibody, multispecific antibody (e.g., bispecific antibody), or fragment thereof provided that it specifically binds to the cell surface marker. Antibodies may be obtained by standard techniques comprising immunizing an animal with a target antigen and isolating the antibody from serum. Monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The monoclonal antibodies may also be isolated from phage antibody libraries using the techniques described in Clackson et al., Nature 352:624-628 (1991) and Marks et al., J. Mol. Biol. 222:581-597 (1991), for example. The antibody may also be a chimeric or humanized antibody. Many antibodies against cell surface markers are commercially available and well known in the art, e.g. trastuzumab (Herceptin®) which binds to HER-2.

Two general methods of IHC are available; direct and indirect assays. According to the first assay, binding of antibody to the target antigen is determined directly. This direct assay uses a labelled reagent, such as a fluorescent tag or an enzyme-labelled primary antibody, which can be visualized without further antibody interaction.

In a typical indirect assay, unconjugated primary antibody binds to the antigen and then a labelled secondary antibody binds to the primary antibody. Where the secondary antibody is conjugated to an enzymatic label, a chromagenic or fluorogenic substrate is added to provide visualization of the antigen. Signal amplification occurs because several secondary antibodies may react with different epitopes on the primary antibody.

The primary and/or secondary antibody used for immunohistochemistry typically will be labeled with a detectable moiety. Numerous labels are available, including radioisotopes, colloidal gold particles, fluorescent labels and various enzyme-substrate labels. Fluorescent labels include, but are not limited to, rare earth chelates (europium chelates), Texas Red, rhodamine, fluorescein, dansyl, Lissamine, umbelliferone, phycocrytherin and phycocyanin, and/or derivatives of any one or more of the above. The fluorescent labels can be conjugated to the antibody using known techniques.

Various enzyme-substrate labels are available, e.g. as disclosed in U.S. Pat. No. 4,275,149. The enzyme generally catalyzes a chemical alteration of the chromogenic substrate that can be detected microscopically, e.g. under visible light. For example, the enzyme may catalyze a colour change in a substrate, or may alter the fluorescence or chemiluminescence of the substrate. Examples of enzymatic labels include luciferases (e.g. firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are well known.

Horseradish peroxidase may be visualised with hydrogen peroxidase as a substrate, wherein the hydrogen peroxidase oxidizes a dye precursor (e.g. orthophenylene diamine (OPD) or 3,3′,5,5′-tetramethyl benzidine hydrochloride (TMB). Alkaline phosphatase (AP) may be detected with para-nitrophenyl phosphate as chromogenic substrate. β-D-galactosidase (β-Gal) may be detected with the chromogenic substrate p-nitrophenyl-β-D-galactoside or fluorogenic substrate 4-methylumbelliferyl-β-D-galactoside.

In an embodiment of the present invention, the immunohistochemistry step may be performed as follows. Following an optional blocking step, the tissue section is exposed to primary antibody for a sufficient period of time and under suitable conditions such that the primary antibody binds to the cell surface marker in the tissue sample. Appropriate conditions for achieving this can be determined by routine experimentation. The tissue sample is then exposed to a secondary antibody which binds specifically to the primary antibody (e.g. the primary antibody is a mouse monoclonal antibody and secondary antibody is a rat anti-mouse polyclonal antibody). The cell surface marker can then be visualised by applying to the sample a chromogenic substrate for an enzyme conjugated to the secondary antibody.

Specimens thus prepared may be mounted and coverslipped. The stained sample is now ready to be imaged for subsequent analysis.

Obtaining an Image of the Stained Sample

In embodiments of the present invention, the stained sample (e.g. stained tissue section) is analysed by first obtaining an image of the sample. Typically the image is obtained under magnification, e.g. 20× magnification, for instance using a microscope. In a preferred embodiment the image is obtained by an automated image acquisition system, e.g. an apparatus capable of automatic scanning of prepared microscope slides. The imaging system may additionally be capable of automated image analysis.

Suitable automated imaging systems for use in the present methods are disclosed in, for example, U.S. Pat. No. 6,718,053, U.S. Pat. No. 7,233, 340, U.S. Pat. No. 7,177,454, U.S. Pat. No. 6,215,892, U.S. Pat. No. 6,631,203, U.S. Pat. No. 7,272,252, US 2006/0072805, WO 2008/023055, US 2004/0085443, U.S. Pat. No. 7,171,030, U.S. Pat. No. 6,466,690, U.S. Pat. No. 6,917,696, U.S. Pat. No. 7,457,446 and U.S. Pat. No. 7,518,652.

Typically such an automated imaging system comprises an optical microscope, a digital camera (e.g. comprising a CCD array) and a motorized stage. The system may further comprise a slide carrier or other device for loading slides onto the stage. Digital images of different areas of the sample can be acquired and stored on a suitable storage means. In some embodiments, a scanner or other image capture device may be used in place of a digital camera.

Typically the system is controlled by a computer comprising a processor executing instructions from a computer program residing on a computer-readable medium. The computer may control, for example, loading of slides onto the stage, selection of regions of interest on the slides by movement of the stage, operation and focusing of the microscope, image acquisition by the camera and storage of the acquired images. The images may be stored on any computer-readable medium such as a hard disk, computer-readable CD, RAM etc.

In some embodiments, each slide may be labelled with a bar code, which may be read by a bar code reader within the slide loader in the imaging system. A user may set analysis parameters according to the information recorded in the bar code, by inputting settings into the computer. For instance, the bar code may indicate that the slide has been labelled with a particular antibody/fluorochrome, such that fluorescent image capture by the microscope needs to operate at a particular wavelength. The bar code may also include information identifying the source of the sample, for example an identifier for the patient/tissue from which the sample was taken.

An automated focusing routine may be performed at each position, based on a contrast search through the slide in the Z direction. This involves varying the Z position of the stage and identifying a position of maximum contrast. A coarse focus may be performed first, followed by a fine focus. Automated focusing methods are disclosed, for example, in U.S. Pat. No. 6,215,892.

Various filters may be used during image acquisition. Typically red, green and blue filters are used such that 3 images of the slide are obtained at each stage position. The 3 RGB images are then assembled into a single colour image for that position. Images and associated data may be stored to a database, which can be local to the scan station computer system or remotely hosted on a server.

The multiple image fields obtained from the low power scan may be assembled to form a single image of the slide, which may be displayed on the computer screen. The system may be configured to automatically analyse the low magnification image in order to identify regions of the slide containing a tissue sample. Alternatively, a user may manually select a region of the slide for further analysis by interaction with the computer. The computer may be configured to recognise areas of interest labelled on the slide using, for example, a marker pen. The system may also be capable of recognising multiple tissue samples positioned in an array on a single slide, for example in the case of tissue microarray slides. Multiple slides containing sections cut from the same tissue block may be scanned and “linked” such that regions defined on one of the slides may be propagated to other slides.

Similar image acquisition routines may be performed for both fluorescent and brightfield imaging, except that fluorescent imaging involves excitation and emission at specific wavelengths. The end result of this process is a high magnification image of the areas of interest on the stained sample.

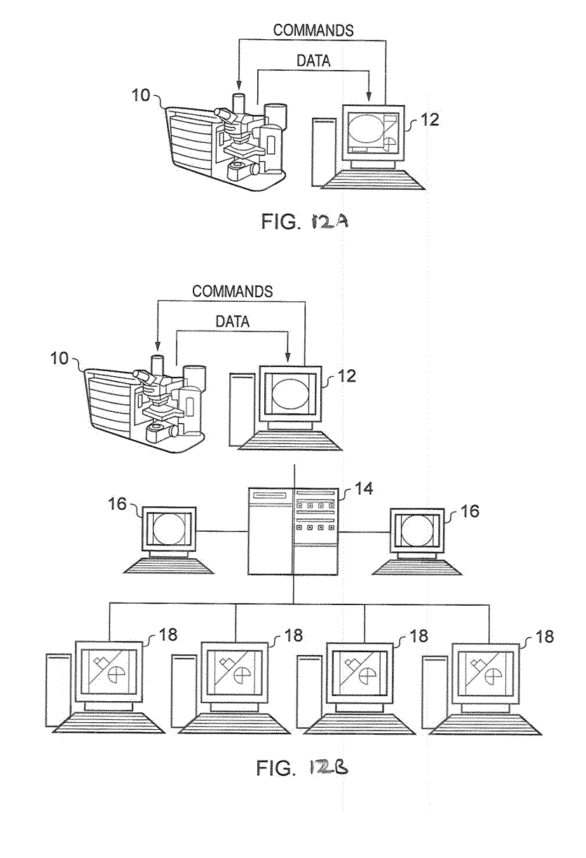

FIG. 12A schematically illustrates one example microscope system for capturing images of a sample for use in accordance with embodiments of the invention. The microscope unit 10 captures digital images of a sample under investigation and the digital images are transferred to computer 12 where they are stored. In this example the computer is a suitably programmed general purpose computer coupled to the microscope unit in a conventional manner. In other examples the computer may be an application specific device for the application at hand. Furthermore in some embodiments the microscope unit and computer may be integrated into a single apparatus. Furthermore still, in addition to operating in conjunction with the microscope unit to acquire images, in some embodiments the computer may also perform image processing of acquired images in accordance with embodiments of the invention, e.g. as described further below.

The microscope unit 10 in this example can illuminate with white light for the capturing of bright field digital images, and can also illuminate with a range of specific wavelengths by means of a filter set for the excitation of particular fluorescent emissions.

In some embodiments a slide holding the sample may be loaded manually by a user, but in the illustrated example the microscope unit 10 comprises a set of microscope slide racks and an automated slide loader, so that a series of slides may be selected, positioned under the microscope, imaged and returned to the slide racks.

Furthermore, in the illustrated embodiment the computer 12 sends commands to the microscope unit 10 dictating which slides should be imaged, what magnifications they should be imaged at, which light source should be used to illuminate each slide, and so on, in accordance with desired imaging characteristics. Once a series of captured images has been transferred from the microscope unit 10 to the computer 12, they may be further processed/analyzed. This may be done, for example, by an automated processing algorithm running on the computer 12 or connected device, or by a user operating the computer 12 (or connected device). The example system illustrated in FIG. 12A is the Ariol® imaging system produced by Applied Imaging/Genetix.

FIG. 12B schematically illustrates the microscope system of FIG. 12A connected to a server 14 and a network. The network consists both of computing devices 16 connected locally to the server 14, and of computing devices 18 located remote from the server 14, for example in a local area network (LAN) or via the internet. In the example arrangement illustrated in FIG. 12B the captured images taken by the microscope unit 10 are uploaded from the computer 12 to the server 14, such that any of the other computing devices 16 or 18 connected to the server 14 may also view those captured images, perform analysis on them etc.

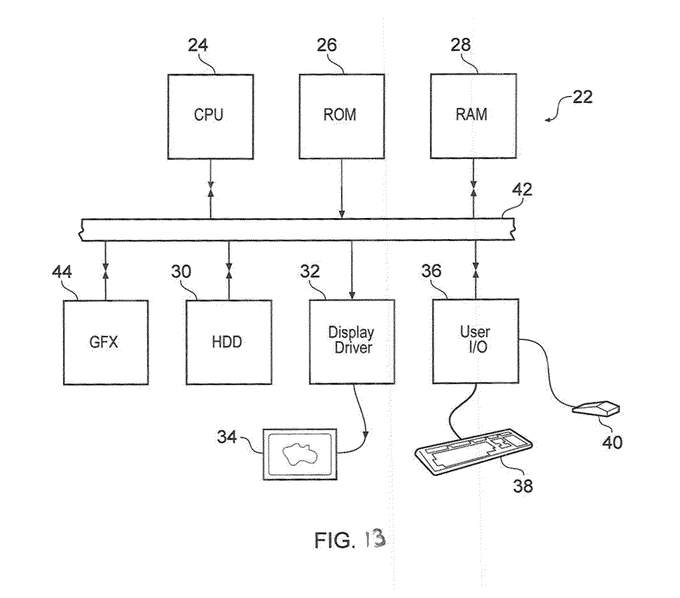

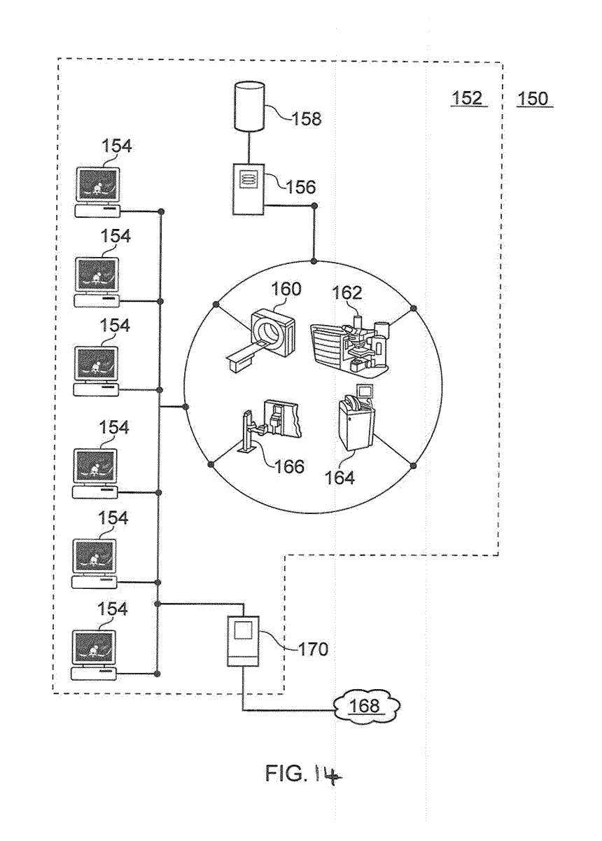

FIG. 13 schematically illustrates a general purpose computer system 22 (such as any of computers 12, 16 or 18 in FIGS. 12A and/or 12B) configured to perform processing of captured images in accordance with an embodiment of the invention. The computer 22 includes a central processing unit (CPU) 24, a read only memory (ROM) 26, a random access memory (RAM) 28, a hard disk drive (HDD) 30, a display driver 32 and display 34, and a user input/output (I/O) circuit 36 with a keyboard 38 and mouse 40. These devices are connected via a common bus 42. The computer 22 also includes a graphics card 44 connected via the common bus 42. The graphics card includes a graphics processing unit (GPU) and random access memory tightly coupled to the GPU (GPU memory) (not shown in FIG. 13). In other examples the computer system may not include a dedicated GPU.

The CPU 24 may execute program instructions stored in the ROM 26, in the RAM 28 or on the hard disk drive 30 to carry out processing of captured images, for which associated data may be stored within the RAM 28 or the hard disk drive 30. The RAM 28 and hard disk drive 30 may be collectively referred to as the system memory. The GPU may also execute program instructions to carry out processing of captured image data passed to it from the CPU.

FIG. 14 shows an example computer network which can be used in conjunction with embodiments of the invention. The network 150 comprises a local area network in a hospital 152. The hospital 152 is equipped with a number of workstations 154 which have access, via a local area network, to a hospital computer server 156 having an associated storage device 158. A PACS (Picture Archiving and Communications System) archive is stored on the storage device 158 so that data, e.g. image data from a microscope unit such as shown in FIGS. 12A and 12B, in the archive can be accessed from any of the workstations 154 for processing in accordance with embodiments of the invention. One or more of the workstations 154 is operable to process image data in accordance with embodiments of the invention as described hereinafter. Software/processing instructions for configuring the workstations to process images in accordance with embodiments of the invention may be stored locally at each workstation 154, or may be stored remotely and downloaded over the network 150 to a workstation 154 when needed. Also, a number of medical imaging devices 160, 162, 164, 166 are connected to the hospital computer server 156 and imaging data collected with the devices 160, 162, 164, 166 can be stored directly into the PACS archive on the storage device 156. Of particular interest in the context of the present invention are the captured images from microscope unit 162, which unit may be similar to the microscope unit shown in FIG. 12A and described above. The local area network is connected to the internet 168 by a hospital internet server 170, which allows remote access to the PACS archive. This is of use for remote accessing of data and for transferring data between hospitals, for example, if a patient is moved, or to allow external research to be undertaken.

It will be appreciated that images suitable for use in conjunction with embodiments of the invention may be obtained using any suitable instrumentation technology, e.g. based on line scanner technologies.

Determining Continuity of Cell Surface Staining

In embodiments of the present invention, the method comprises a step of determining a value for continuity of cell surface staining in the image. For example, the image is analysed in order to quantify the continuity of membrane staining associated with the marker. This analysis may be performed by an automated imaging system as discussed above.

Typically the method comprises a step of detecting stained regions within the image. Pixels in the image corresponding to staining associated with the marker of interest may be identified by colour transformation methods, for instance as disclosed in U.S. Pat. No. 6,553,135 and U.S. Pat. No. 6,404,916. In such methods, stained objects of interest may be identified by recognising the distinctive colour associated with the stain. The method may comprise transforming pixels of the image to a different colour space, and applying a threshold value to suppress background. For instance, a ratio of two of the RGB signal values may be formed to provide a means for discriminating colour information. A particular stain may be discriminated from background by the presence of a minimum value for a particular signal ratio. For instance pixels corresponding to a predominantly red stain may be identified by a ratio of red divided by blue (R/B) which is greater than a minimum value.

The transformed image may be further analysed to determine the presence of structures of interest, in this case positively stained cell surfaces, by grouping together pixels in close proximity and having the same colour. Edge detection techniques may be applied to discriminate the cell membrane from other structures. In some embodiments cells may be identified, for example, by identifying nuclei stained with a counterstain. The computer may locate and count such nuclei by detecting the image intensity in a channel associated with the counterstain. Positive staining corresponding to the cell surface marker on these cells may detected by measuring a particular colour in close proximity to a counterstained nucleus, e.g. a brown colour indicative of a stain used to visualise an anti-HER-2 antibody may be detected surrounding each nucleus which is stained blue with a stain such as DAPI.

Such an analysis identifies regions of the image corresponding to cell surfaces or membranes, and classifies individual pixels as either positive or negative for the stain which labels the marker. In the next step, the method may comprise determining the continuity of cell surface staining from these parameters. By “continuity of cell surface staining” it is meant the extent to which regions of the image corresponding to cell surfaces (e.g. membranes) are continuously stained, for example whether staining is uninterrupted around the entire circumference of the cells or whether there are gaps between regions of stained membrane. The continuity of membrane staining may be assessed by determining the degree to which there are adjacent or connected pixels having the characteristic stain colour within regions of the image corresponding to the cell surface. For instance, the percentage of the cell membrane which is continuously stained (i.e. comprises connected positive pixels) may be determined.

In one embodiment, the method may comprise a further step of determining the intensity of cell surface staining By “intensity of cell surface staining” it is meant that the overall level of staining within cell surface regions of the image is determined, for instance by determining an absorbance value for the membrane regions and/or the percentage of pixels positive for the stain in the membrane.

In one embodiment, the cell surface staining continuity value may be used to provide an indicator of the expression level of the cell surface marker in the sample. In a preferred embodiment, the continuity value is combined with the intensity value in order to indicate the expression level. For instance, the continuity value and intensity value may be combined to provide a weighted probability value indicative of a probability of the sample being classified in a predefined staining class.

In one particularly preferred embodiment, the cell surface marker is HER-2 and the method may be performed in order to classify a sample into a standard staining class, for example according to the ASCO/CAP and UK guidelines. In this method, the sample is typically classified into one of the nominal categories 0, 1+, 2+ or 3+.

In one embodiment, the probability that a given sample should be classified in a particular category is provided by use of the probability distribution function,

$$\begin{matrix}{P}_{( x )} = \frac{1}{\sqrt{2 π} {σ}_{( x )}} exp ( - \frac{{( x - {μ}_{( x )} )}^{2}}{2 {σ}_{( x )}^{2}} ) & ( 1 ) \\ {P}_{( t )} = {P}_{( abs )} * {P}_{( cont )} * {P}_{( cont )} & ( 2 )\end{matrix}$$

wherein:

X=variable associated with the sample image (e.g. absorbance (abs) or continuity (cont))

P₍ₓ₎=probability associated with variable X

μ₍ₓ₎=an average for variable X

σ₍ₓ₎=a standard deviation for variable X

π=3.1417

Aspects of the invention may be implemented in hardware or software, or a combination of both. However, preferably, the methods of the invention are implemented in one or more computer programs executing on a programmable processor in a computer or imaging apparatus as described herein. The computer or apparatus may further comprise at least one data storage system (including volatile and non-volatile memory and/or storage elements), at least one input device, and at least one output device. Program code is applied to input data to perform the functions described herein and generate output information. The output information is applied to one or more outputs, in known fashion.

Each program may be implemented in any desired computer language (including machine, assembly, high level procedural, or object oriented programming languages) to communicate with a processing system. In any case, the language may be a compiled or interpreted language. Each such program is preferably stored on a storage media or device (e.g., ROM, CD-ROM, tape, or magnetic media) readable by a general or special purpose programmable processor, for configuring and operating the processor when the storage media or device is read by the processor to perform the procedures described herein. The inventive system may also be considered to be implemented as a computer-readable storage medium, configured with a computer program, where the storage medium so configured causes a processor to operate in a specific and predefined manner to perform the functions described herein.

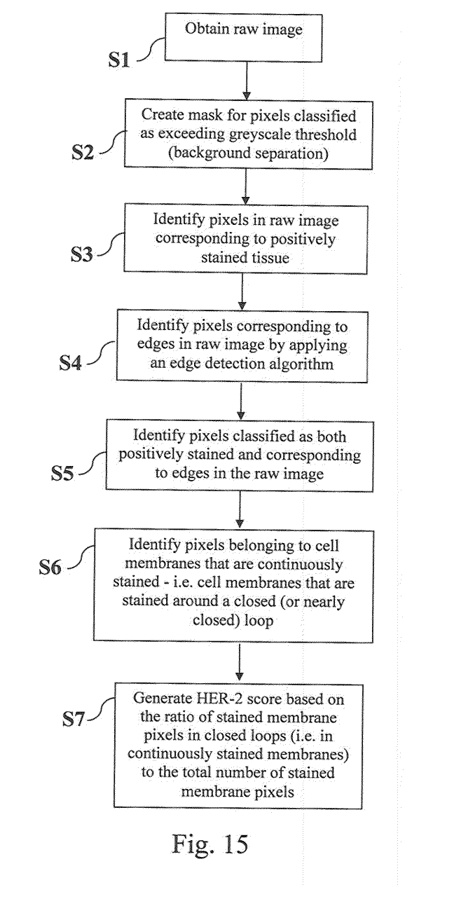

Thus FIG. 15 schematically represent a method of processing an image in accordance with an embodiment of the invention. The method may be implemented by a conventional computer operating under control of appropriately configured software. In this example the method is applied to a conventional colour-stained IHC digital microscopy image of a tissue sample of interest which has been obtained in a conventional manner at a magnification of 20×.

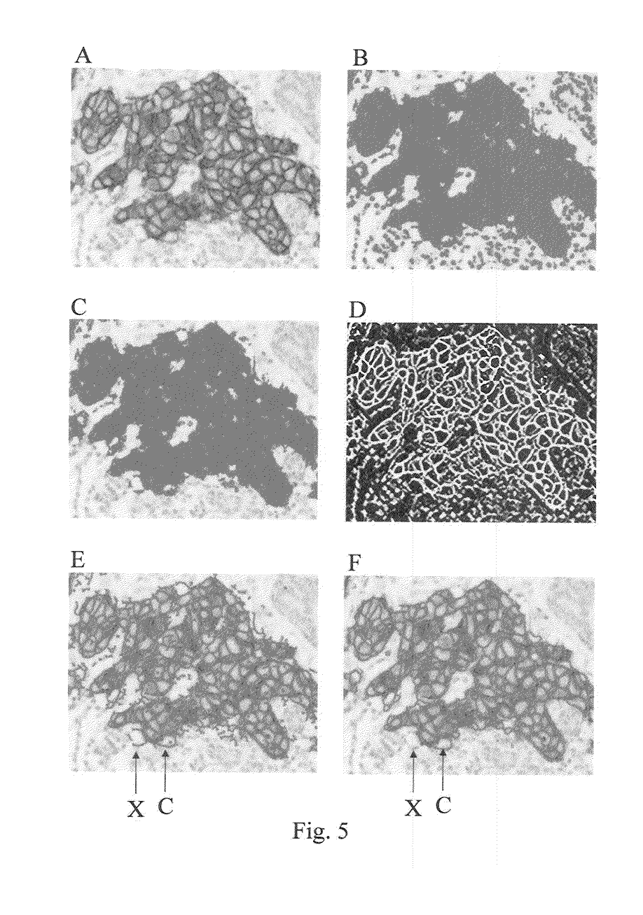

FIG. 5A schematically shows a representation of an example colour-stained IHC image of a tissue sample which may be processed in accordance with embodiments of the invention. As is conventional, the image of FIG. 5A is represented by a data set that defines the imaged characteristic (i.e. colour) over an array of pixels that may be spatially mapped to the sample tissue. In this example, the image is obtained for a conventional brown-stained IHC tissue sample using a conventional Digital Slide scanner. Each pixel is associated with a colour value defined by three parameters. As is well known, there are many ways of defining a colour value for a pixel in a digital image. Here it is assumed the Red-Green-Blue (RGB) model is used for defining a pixel's colour value in colour space. Other schemes (e.g. based on a Hue-Saturation-Intensity (HSI) parameterisation) could equally be used. Colour values are thus defined by the three parameters R, G and B. The R, G and B values may, for example, be parameterized such that each runs from 0 to 255. A colour of a pixel may thus be represented by a position in a three-dimensional colour space defined by R, G and B axes.

In the below-described example of processing in accordance with the method of FIG. 15 the aim of the processing is to derive a parameter indicative of a HER-2 score that based on the degree of positive (i.e. brown) staining in cell membranes of the sample image represented in FIG. 5A, and in particular on the extent to which the cell membranes are considered to be continuously (completely) stained.

Thus referring to FIG. 15, in Step S1 a conventionally colour-stained IHC image of a tissue sample is obtained. This may be obtained directly from a digital imaging microscope (e.g. microscope unit 10 of FIG. 12A), or from a database/store or previously obtained images (e.g. from storage device 158 of FIG. 14). As noted above, FIG. 5A shows an example of such an image. The image of FIG. 5A may be referred to in the following as an initial or raw image. However, it should be noted this terminology is used for convenience and is not intended to preclude the prior use of any pre-processing steps which may conventionally be applied to IHC images.

In Step S2 a threshold mask image is created to distinguish pixels in the raw image considered to correspond to the tissue sample from those which are considered to correspond to background. This is achieved on the basis of conventional thresholding applied to a greyscale representation of the raw image. Thus the RGB colour values for each pixel are converted to a greyscale value, e.g. using conventional techniques such as provided by the ImageJ Java-based image processing library developed by the US National Institutes of Health. In this example colour values are converted to 8-bit greyscale values (i.e. 0-255) and compared with a threshold intensity value Tₜₕᵣₑₛₕ. Pixels having a greyscale intensity value above or equal to the threshold intensity value Tₜₕᵣₑₛₕ are considered to be associated with the tissue sample of interest. These pixels may sometimes be referred to here as foreground pixels. Pixels having a greyscale intensity value less than the threshold intensity value Tₜₕᵣₑₛₕ are considered to be associated with background. These pixels may sometimes be referred to here as background pixels.

A threshold intensity value Tₜₕᵣₑₛₕ of around 230 has been found to be suitable in embodiments of the invention. However other values may be used. For example, different values may be appropriate for images obtained for different imaging conditions, e.g. different exposure times. An appropriate value for particular imaging conditions may be based on a previously performed training step in which a user analyses the results from processing generally in accordance with FIG. 15 but using different threshold intensity values. A threshold intensity value providing suitable results may then be selected by the user and used for processing sample images obtained under conditions corresponding to those of the training image.

In accordance with known techniques, the binary classification as to whether a pixel is considered to be associated with tissue of interest or background may be represented by defining a threshold mask image. The threshold mask image comprises an array of pixels corresponding to the image under analysis. Each pixel in the threshold mask image is associated with a binary classification parameter—e.g., a zero value if the corresponding pixel location in the raw image is considered to be associated with background (i.e. a greyscale value less than the threshold intensity value Tₜₕᵣₑₛₕ), and a unit value if the corresponding pixel location in the raw image is considered to be a foreground pixel.

FIG. 5B schematically shows the image of FIG. 5A overlain with a threshold mask image determined according to Step S2 of the method of FIG. 15. The threshold mask image is displayed as green where the corresponding pixels of FIG. 5A are considered foreground pixels (based on the greyscale thresholding of Step S2) and transparent elsewhere. A comparison of FIG. 5B with 5A shows that Step S2 identifies the majority of the central region of the image corresponding to the brown staining seen in FIG. 5A as being foreground pixels and also a number of surrounding smaller regions associated with tissue that is not stained brown.

In Step S3 the pixels in the raw image are classified according to whether or not they are considered to be associated with regions in the sample which are positively stained. This is based on comparing the colour values for each pixel with a predefined range of colour values deemed to be associated with the colour of the relevant stain (in this example brown stain). In this example this is only done for the pixels shown green in FIG. 5B—i.e. the foreground pixels identified at Step S2.

Thus in Step S3 each foreground pixel in the raw image is classified according to whether its colour value (as defined by the colour space parameters being used—in this example R, G, and B) falls within an expected range for positively-stained tissue, e.g. as defined in a colour definition file. The expected range for a given application will depend on characteristics of the tissue sample and staining method used.

A suitable colour definition file to achieve this classification comprises a 3D array with axes corresponding to possible R, G and B values. For each location in this 3D array (i.e. for each combination of R, G and B) a binary index value is specified—e.g. unity if the associated RGB values define a colour that is considered to be associated with positive staining and zero if the associated RGB values define a colour that is not considered to be associated with positive staining.

Thus the index value in the colour definition file for the RGB values for each foreground pixel in the raw image identify whether or not the pixel is associated with positive staining The specification of the colour definition file (i.e. the setting of the index value for each RGB combination) may be based on an initial previously-performed training step. Thus a user may be provided with a characteristic training image for which he manually identifies a range of pixels which he considers to be positively stained for the application at hand. The RGB values for the identified pixels, and interpolations between these colours, may thus be used to populate the index values of a colour definition file in an appropriate manner. Once this has been done for the training image, the same colour definition file may be used for processing other sample images.

In principle the colour definition file may include an entry for all possible colours, that is to say for all possible 256×256×256 RGB combinations. However, in practice binning of R, G and B values may be used to reduce the size of the array. For example the possible 256 values for each colour axis may be binned down to 12 values (0 to 11) per axis.

As with the threshold mask discussed above, the binary classification as to whether a pixel is considered to be positively stained tissue (as determined in Step S3 applied to the foreground pixels) may be represented by defining a mask image, which may conveniently be referred to as a positively-stained mask image. Thus the positively-stained mask image comprises an array of pixels corresponding to the image under analysis. Each pixel in the positively-stained mask image is associated with a binary classification parameter—e.g., a unit value if the corresponding pixel location in the raw image is considered to be associated with a positively stained foreground pixel and zero otherwise.

FIG. 5C schematically shows the image of FIG. 5A overlain with a positively-stained mask image determined according to Step S3 of the method of FIG. 15. The positively-stained mask image is displayed as green where the corresponding pixels of FIG. 5A are considered positively stained foreground pixels (based on the greyscale thresholding of Step S2 and colour-based discrimination of Step S3) and transparent elsewhere. A comparison of FIG. 5C with 5A shows that Step S3 identifies the majority of the central region of the image corresponding to the brown staining seen in FIG. 5A as being positively stained foreground pixels. The surrounding smaller regions seen of the threshold mask seen in FIG. 5B are not present in the positively-stained mask of FIG. 5C since these areas do not match the positive stain colour.

In Step S4 pixels corresponding to object edges/boundaries in the raw image are identified using a conventional edge detection algorithm. In one example the edge detection algorithm may be based on the “convolve” function of the ImageJ image processing library with a 13-element 1D kernel applied separately along each direction for each colour of the 2D raw image. A suitable kernel for some implementations might comprise the elements {−3, −3, −2, 2, 2, 3, 3, 2, 2, 2, −2, −3, −3}, for example. The results from applying the kernel in the two directions are then combined to form an image representing the identified edges in the raw image. For example the combining may be based on selecting for each pixel location the highest of the two values obtained by applying the convolve function in the two different directions.

FIG. 5D is an image schematically showing the results of applying the edge detection algorithm of Step S4 to the raw image of Step S1. In this example the edge detection algorithm is applied separately to the R, G and B values for the pixels comprising the image and the resulting three images are combined to form a what might be seen as colour image of the edges. However in other examples the edge detection may be applied to a grayscale representation of the image of FIG. 5A (e.g. based on grayscales similar to those used in Step S2 described above). Furthermore, in still other examples edge detection may be based on only one vector of the colour space—e.g. by applying the edge detection algorithm to the red values for the pixels.

Referring back to FIG. 5D, the region in the middle of the image corresponding to the area of brown staining in FIG. 5A clearly show the edges associated with the cell membranes. In addition to this a “noise”-like pattern is seen in the surrounding region associated with the structures in the image that are not brown stained.

In Step S5 pixels identified as being both positively stained in Step S3 and part of an object edge/boundary in Step S4 are identified. These pixels are taken to correspond to positively stained regions of cell membrane in the raw image. As with the threshold and positively-stained masks discussed above, the binary classification as to whether a pixel is considered to be both positively stained tissue (as determined in Step S3) and an edge (based on the results of the edge detection in Step S4) may be represented by defining a positively-stained membrane mask image. The positively-stained membrane mask image comprises an array of pixels corresponding to the image under analysis. Each pixel in the positively-stained membrane mask image is associated with a binary classification parameter—e.g., a unit value if the corresponding pixel location in the raw image is considered to correspond to a positively-stained region of cell membrane (based on the results of Steps S3 and S4) and zero otherwise. A pixel may be classified as being at an object edge/boundary if the corresponding pixel in the image resulting from the application of the edge detection process has a value exceeding a given threshold. In this example a pixel is considered to comprise an edge if the application of the edge detection in Step S4 to the blue channel provides a value greater than a predefined edge threshold. An edge threshold of around 100 has been found to be suitable in some applications. More generally, a suitable value for the edge threshold for other implementations may be based on a training step similar to those described above in which a user manually assesses the performance of the processing of FIG. 15 to determining a value that provides acceptable/optimum results. This value can then be used for other images.

A filter may be applied to the positively-stained membrane mask, e.g. a median filter.

FIG. 5E schematically shows the image of FIG. 5A overlain with a positively-stained membrane mask image determined according to Step S5 of the method of FIG. 15. The positively-stained membrane mask image is displayed as green where the corresponding pixels of FIG. 5A are considered positively-stained membrane pixels (based on results of Step S5) and transparent elsewhere. A comparison of FIG. 5E with 5A shows that Step S5 clearly identifies the regions of stained cell membrane apparent as dark brown staining in FIG. 5A.

In Step S6 the positively-stained membrane mask is used to identify individual cell membranes that are continuously (or nearly continuously) stained—i.e. those cell membranes that are stained around a closed (or nearly closed) loop in the image of FIG. 5A. The classification as to whether pixels are considered part of a membrane that is continuously stained may again be represented by defining a mask image, which may conveniently be referred to as a continuously-stained membrane mask image. In broad summary the continuously-stained membrane mask is obtained by starting from the positively-stained membrane mask and identifying end points in the morphology of positively stained membrane. Where a pair of endpoints is separated by only a small gap (e.g. where a cell membrane is nearly continuously stained but with a small non-stained region), the gap is in effect filled to form a closed loop. The aim of this part of the processing is to account for small gaps which result from artefacts of the image acquisition/processing rather than from real gaps in the stained membrane. Parts of the positively-stained membrane mask which comprise isolated endpoints (i.e. not near to another endpoint) are removed from the mask for not being part of a continuously-stained membrane. In this example the processing of Step S6 is achieved in various stages.

In a first stage the edges represented in the positively-stained membrane mask are thinned, e.g. using the ConvolveBinary function available at http://voxel.jouy.inra.fr/darcs/imagej-mima2/externalPlugins/Morphology/BinaryThin2_.java.

In a second stage gaps between pairs of endpoint are closed if the gaps are smaller than a threshold size, e.g. less than seven pixels in one example embodiment. That is to say pixels between the gaps in the thinned positively-stained membrane mask are set to unity in the continuously-stained membrane mask. In this example each endpoint may only be joined to one other. Furthermore the continuously-stained membrane mask image may be dilated, e.g. using a conventional algorithms, to fill other gaps and then thinned. This can also aid in accounting for small gaps which result from artefacts of the image acquisition/processing rather than from real gaps in the stained membrane. Appropriate characteristics for such thinning/dilation when applied can again be determined based on observing the results of the processing applied to a “training” image using different parameters.

In a third stage endpoints in the continuously-stained membrane mask that have not been joined are pruned away, e.g. using conventional image processing/eroding techniques.

The resulting continuously-stained membrane mask may then be dilated so that its features are more easily viewed.

Thus the continuously-stained membrane mask provides a binary classification as to whether or not stained pixels in the raw image are considered part of a closed (i.e. complete) cell membrane or not.

For example, referring to FIG. 5E a partially-stained cell membrane is identified by the arrow labelled X and a continuously stained cell membrane is identified by the arrow C. The gaps of non-staining in cell membrane X are too large to be closed (i.e. the gaps are above the 7-pixel deminimis threshold of this example) and so these sections of stained membrane are removed in Step S6 in forming the continuously-stained membrane mask from the positively-stained membrane mask (which represents both continuously and partially stained membrane tissue).

FIG. 5F schematically shows the image of FIG. 5A overlain with a continuously-stained membrane mask image determined according to Step S6 of the method of FIG. 15. The continuously-stained membrane mask image is displayed as green where the corresponding pixels of the positively-stained membrane mask of FIG. 5E are considered part of a continuously-stained cell membrane (based on the results of Step S6) and transparent elsewhere. A comparison of FIG. 5F with 5A shows that Step S6 clearly identifies a series of closed loops that map well to the regions of cell membrane apparent in the middle region of FIG. 5A and which can be seen to be continuously stained (i.e. positively stained around their complete periphery in the image).

As discussed above, a significant aspect of embodiments of the present invention is a measure of the extent of to which cell membranes are continuously stained (i.e. the extent to which the staining that is present in the image is in closed loops). This can be parameterised based on the results of the processing of FIG. 15. For example the number of pixels shown green in FIG. 5F is a measure of the number of pixels deemed to correspond to cell membranes which are continuously stained (i.e. forming closed loops). The number of pixels shown green in FIG. 5E, on the other hand, is a measure of the number of pixels deemed to correspond to positively stained cell membrane tissue, regardless of whether the individual membranes are continuously of only partially stained. The ratio of these two numbers (referred to here as “% membrane continuity”) is a measure of the percentage of membrane that is continuously stained (i.e. the fraction of membrane staining that forms closed loops). This may be used in determining an Her-2 score as discussed above. Other parameters relating to the extent of staining may also be employed in computing an HER-2 score. For example. as well as taking account of the percentage of continuously stained membrane, the overall percentage of positively stained pixels which are considered to correspond to membrane tissue (e.g. based on the ratio of the number of pixels identified in the positively-stained membrane mask of FIG. 5E to the number of pixels identified in the positively-stained mask of FIG. 5C) and/or a measure of membrane staining absorbance may be used.

Thus in step S7 a value for % membrane continuity is determined from the masks represented in FIGS. 5E and 5F and used to predict an HER-2 score for the sample. In this example this is done by first determining the probabilities for a sample displaying the computed % membrane continuity being associated with the various HER-2 scores. This is done in accordance with the general formula presented above (Equation 1) where X is % membrane continuity and with values for μ₍ₓ₎, σ₍ₓ₎, as set out in the examples below for the different HER-2 scores under the heading “Probability Model”. A predicted HER-2 score is then determined based on the determined probabilities for the different HER-2 scores, again as set out below under the heading “Probability Model”.

However, as can be seen from these examples, for this embodiment the probability of a sample having a particular HER-2 score is not based solely on % membrane continuity, but also takes account of a parameter representing the membrane staining absorbance. In this example the membrane staining absorbance (referred to here as “absorbance”) is based on an average level of grayscale intensity for the pixels identified in Step S5 as belonging to cell membranes (i.e. the pixels shown green in FIG. 5E). In particular, in this embodiment a histogram of the grayscale intensity for these pixels if formed (using a histogram bin-width of 8 counts for smoothing). The grayscale value at the centre of the bin containing the most pixels (i.e. the modal average) is taken to represent an average intensity for the membrane pixels. An absorbance parameter is then determined according to the following:

$$absorbance = - ln \frac{MembraneIntensity}{{T}_{thresh}} * 100$$

where Tₜₕᵣₑₛₕ is the threshold parameter used at step S2.

Thus in Step S7, and as shown in the worked examples below, a first parameter P_{(cont)} is determined by applying Equation 1 to the variable X=% membrane continuity with relevant values for μ₍ₓ₎, σ₍ₓ₎, as set out in the examples below for the different possible HER-2 scores. In addition a second parameter P_{(abs)} is determined by applying Equation 1 to the variable X=absorbance, again with relevant values for μ₍ₓ₎, σ₍ₓ₎, as set out in the examples below for the different possible HER-2 scores.

The resulting values may be combined to derive a parameter P₍ₜ₎ in accordance with Equation 2 above for of the possible HER-2 scores under consideration.

The values of P₍ₜ₎ may then be combined, again as shown below under the heading “Probability Model” to derive a predicted HER-2 score.

In one example embodiment the percentage of positively stained pixels which are also considered to correspond to membrane tissue (e.g. based on the ratio of the number of pixels identified in the positively-stained membrane mask of FIG. 5E to the number of pixels identified in the positively-stained mask of FIG. 5C) also plays a role in predicting an HER-2 score in that if this percentage is less than a threshold value, e.g. less than 1%, a HER-2 score of 0/1+ is assumed.

It will be appreciate that the processing shown in FIG. 15 represents only one specific example for obtaining suitable parameterisations of the degree of continuous membrane staining in an image and deriving a corresponding HER-2 score. Many other techniques and parameterisation may be used. E.g. the processing of FIG. 15 could readily be modified, for example, to perform steps in a different order, or non-sequentially—e.g. in some example implementations Steps S2 and S3 might be performed simultaneously. Similarly, in some cases a clinical parameter of interest may be derived solely from the extent of continuous membrane staining without taking account of absorbance.

Diagnostic Methods

Using the methods described above, an indication of the expression level of the cell surface marker in the sample may be obtained, e.g. the sample may be assigned a HER-2 staining score. The expression level may have various diagnostic and/or therapeutic applications, particularly where the cell surface marker is associated with disease. For instance, the cell surface marker may be a biomarker, the detection or elevated expression of which is associated with a particular condition. The expression level in the sample determined according to the present method may be compared, for example, to a known standard or to a control sample from a normal subject or tissue, i.e. a sample which is known to be unaffected by the disease or condition.

In one embodiment, the expression level (e.g. HER-2 score) is used in the diagnosis of cancer, e.g. ovarian or breast cancer.

In one embodiment, the cell surface marker is HER-2 and the HER-2 expression level is used to predict responsiveness to therapy with an anti-HER-2 antibody. Typically about 10-30% of breast cancers show an overexpression of HER-2, and may therefore be responsive to anti-HER-2 therapy. Since anti-HER-2 antibodies are both expensive and may induce significant side-effects (such as myocardial toxicity), it is desirable only to treat subjects who are likely to respond thereto. Therefore HER-2 expression level, which may be defined in terms of a HER-2 staining score according to the present methods, may be used in determining whether an anti-HER-2 antibody is administered to a subject. In one embodiment, the anti-HER-2 antibody is trastuzumab (Herceptin®).

### EXAMPLES

Slide Preparations

A total of 448 consecutive cases were selected for this study of which 425 were successfully stained, reviewed and digitized. All cases were formalin fixed and paraffin embedded and were processed in the routine diagnostic laboratory of the institute of origin according to standardised protocols.

Immunohistochemistry

The cases were assessed for HER-2 protein expression using Dako HercepTest® (n=144), Leica Oracle™ HER-2 (n=140) or Ventana Pathway® HER-2 (4b5) (n=141) according to the manufacturer's instructions. In all cases, suitable negative and positive control slides were treated in a similar manner to ensure appropriate staining

Fluorescent in situ Hybridization

A representative cohort of cases was selected for FISH testing for verification purposes. Of the 425 cases supplied, 219 were analysed for HER-2 gene amplification using the PathVysion® HER-2 DNA probe kit and paraffin wax pre-treatment kit (Vysis Inc, UK) in the facility of origin. All procedures were performed in accordance with the manufacturer's recommended protocol.

Digitisation of Slides and Archival of Images

Immunohistochemically stained full face sections were digitised using a NanoZoomer Digital Pathology (NDP) System (Hamamatsu, UK). The NDP system utilises CCD TDI technology to achieve scans with a spatial resolution of 0.46 μm/pixel. Scanning time at 20× was approximately 3 minutes for a 20 mm×20 mm biopsy. Images were approximately 55-487 Mb per whole section biopsy and were archived using Digital Slideserver (SlidePath, Dublin, Ireland), a secure, web-enabled digital slide management system.

### Manual Evaluation of HER-2 Status

In each site, HER-2 protein expression was reviewed and, where appropriate, gene amplification status was also reviewed. For those cases where FISH analysis was carried out, gene amplification status was reviewed by a Biomedical Scientist. All cases were classified according to the new ASCO/CAP and UK guideline recommendations for HER-2 testing as detailed in Table 1.

TABLE 1
ASCO/CAP and UK guideline recommendations for HER-2 classification.^{9, 11}

| Classification | HER-2 Grade | IHC Staining Pattern | FISH Criteria |
|---|---|---|---|
| Negative | 0/1+ | No staining or weak, incomplete | HER2/Chr17 ratio |
| | | membrane staining in <10% of | less than 1.8 |
| | | tumour cells | |
| Equivocal | 2+ | Weak to moderate complete | HER2/Chr17 ratio |
| | | membrane staining that is non- | between 1.8 and 2.2 |
| | | uniform or weak in intensity in at | |
| | | least 10% cells | |
| Positive | 3+ | Uniform, intense membrane | HER2/Chr17 ratio |
| | | staining in >30% of tumour cells. | greater than 2.2 |

Image Analysis Classification of HER-2 Status

Tissue IA System

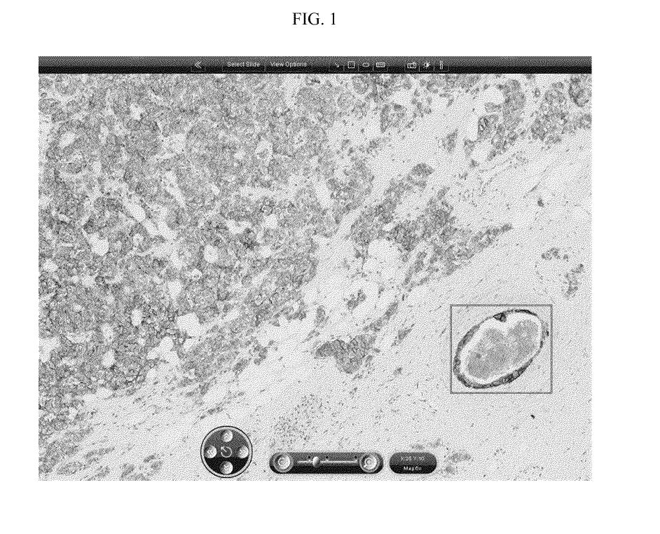

The HER-2 image analysis was performed using an algorithm within the Tissue IA system (SlidePath, Dublin, Ireland), a web-enabled image analysis solution for the interpretation of virtual slides. As a pre-requisite for image analysis, tumour regions of cases were annotated on-line, or where appropriate, regions of DCIS or non-invasive regions were annotated for exclusion (see FIG. 1).

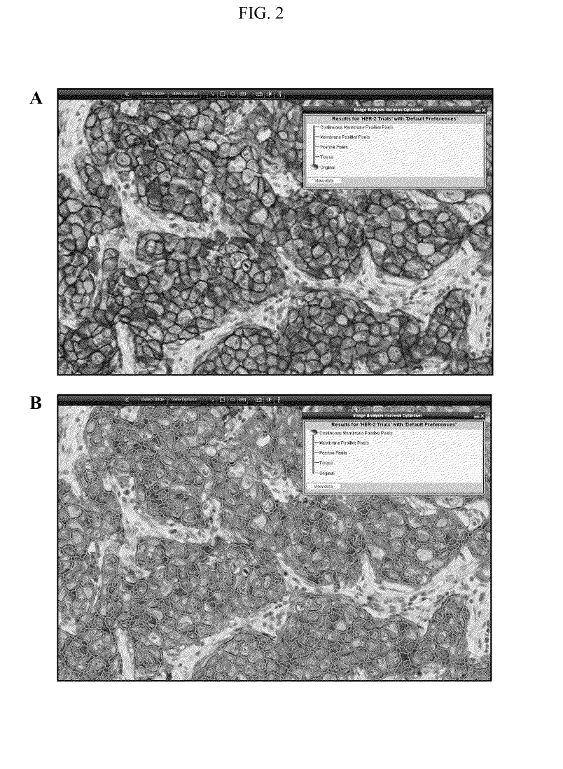

Entire full-face sections or annotated regions of these cases were subsequently submitted for batch image analysis. Tissue IA employs a grid computing model which distributes image data across multiple processing nodes, facilitating high-throughput automated analysis of virtual slides. The HER-2 algorithm utilizes a specific colour definition file to define positively stained tissue within an image and isolates the cell membrane using edge detection techniques (see FIG. 2). The output from the algorithm includes a number of quantitative measurements such as membrane staining absorbance, % membrane positive pixels in tissue and % membrane continuity.

Generation of Probability Classifier

From the total cohort of 425 cases, a training set of 150 cases containing an equal distribution of slides stained with Ventana Pathway®, Leica Oracle™ and Dako HercepTest® antibodies was randomly chosen by assigning cases with a random real number greater than or equal to 0 and less than 1 and selecting the 50 highest numbers for each antibody cohort. Table 2 illustrates the distribution of cases according to the manual review in the training and validation sets.

TABLE 2
Distribution of cases in training and validation sets.

| | Training Set | | Validation Set | |
|---|---|---|---|---|
| | Number of cases | % of Total | Number of cases | % of Total |
| 0/1+ | 89 | 59.4 | 183 | 66.5 |
| 2+ | 32 | 21.3 | 40 | 14.5 |
| 3+ | 29 | 19.3 | 52 | 19.0 |
| Total | 150 | 100 | 275 | 100 |

The image analysis results for these slides were exported for statistical analysis and were used to generate a probability classifier which determined a dedicated HER-2 score (0/1+, 2+ or 3+) based on the distribution of staining absorbance and membrane continuity for each category. In addition, a constraint was included which automatically defined any case with <1% positively stained pixels in selected regions as negative or 0/1+. These computational steps were then incorporated into the algorithm resulting in an output of a dedicated HER-2 classification, along with a percentage confidence in that score.

Probability Model

The algorithm, including the probability classifier generated as described above, was used to classify samples into a HER-2 score category according to the following model:

1. Probability that sample should be classified as Her2 score of 0/1+:

P(0/1)_{absorbance}=(1/(2.50662873*7.586092))*(EXP(−((absorbance-30.58151)²)/(2*(7.586092²))))

P(0/1)_{continuity}=(1/(2.50662873*7.55206))*(EXP(−((% membrane continuity-8.571616))/(2*(7.55206²))))

P(0/1)=P(0/1)_{absorbance}*P(0/1)_{continuity}*P(0/1)_{continuity}

2. Probability that sample should be classified as Her2 score of 2+:

P(2)_{absorbance}=(1/(2.50662873*11.66162))*(EXP(−((absorbance-40.24896)̂2)/(2*(11.66162̂2))))

P(2)_{continuity}=(1/(2.50662873*14.53899))*(EXP(−((% membrane continuity-23.94219)²)/(2*(14.53899²))))

P(2)=P(2)_{absorbance}*P(2)_{continuity}*P(2)_{continuity}

3. Probability that sample should be classified as Her2 score of 3+:

P(3)_{absorbance}=(1/(2.50662873*48.65224))*(EXP(−((absorbance-96.30402)²)/(2*(48.65224²)))

P(3)_{continuity}=(1/(2.50662873*11.90931))*(EXP(−((% membrane continuity-58.90891)²)/(2*(11.90931²))))

P(3)=P(3)_{absorbance}*P(3)_{continuity}*P(3)_{continuity}

4. The output from steps 1 to 3 above is used to generate a provisional HER2 score:

HER2ₚᵣₒᵥᵢₛᵢₒₙₐₗ=IF(AND(P(0/1)>P(2),P(0/1)>P(3)),0/1,IF(AND(P(2)>P(0/1),P(2)>P(3)),2,IF(AND(P(3)>P(0/1),P(3)>P(2)),3)))

That is to say HER2ₚᵣₒᵥᵢₛᵢₒₙₐₗ is set to the HER2 score for which the corresponding value of P (iie, P(0/1), P(2), P(3)) is maximum.

5. A prediction of the HER2 IHC score is provided by the following:

HER2 Grade=IF(AND(% positive pixels<1),0/1,IF(AND(% positive pixels≧1), HER2ₚᵣₒᵥᵢₛᵢₒₙₐₗ))

That is to say, HER2 Grade is set to HER2ₚᵣₒᵥᵢₛᵢₒₙₐₗ unless the parameter “% positive pixels” is less than 1 in which case it is set to 0/1+. The parameter “% positive pixels” is the percentage of positively stained pixels which are also considered to correspond to membrane tissue (e.g. based on the ratio of the number of pixels identified in the positively-stained membrane mask of FIG. 5E to the number of pixels identified in the positively-stained mask of FIG. 5C).

6. The confidence level of the HER2 Predicted IHC Score is provided by the following:

Pₜₒₜₐₗ=SUM(P(0/1), P(2), P(3))

% Confidence 0/1+ score=(P(0/1)/P total)*100

% Confidence 2+ score=(P(2)/P total)*100

% Confidence 3+ score=(P(3)/P total)*100

Validation of Cell-Line Standards

For 180 of the 275 remaining test cases the manufacturer control cell line material was also available for analysis. Cell lines provide consistency in terms of both the quantity of material and the gradation of protein expression, and when used as part of a validated system have applications in internal quality assurance providing a standard against which a laboratory can gauge against day-to-day drift in assay sensitivity.

Statistical Analysis

Statistical analyses including concordance and Cohen's Kappa statistics was performed. The Landis and Koch Kappa interpretation scale was used to evaluate the level of Kappa agreement.

The sensitivity and specificity of both the automated and manual review were calculated using FISH evaluation as the gold standard where:

$$\begin{matrix}Sensitivity = \frac{True Positives}{True Positives + False Negatives} & Equation 1 \\ Specificity = \frac{True Negatives}{True Negatives + False Positives} & Equation 2\end{matrix}$$

Results

Concordance with Manual Review

The concordance between image analysis evaluation of HER-2 status and manual review by a Consultant Pathologist was blindly assessed on a cohort of 275 cases stained with Dako HercepTest®, Leica Oracle™ and Ventana Pathway®. Statistical analysis established that there was agreement in the classification of 250 of the 275 cases, representing a concordance of 91% between the pathology and image analysis reviews (Table 3). Kappa was evaluated to be 0.81, which indicates ‘almost perfect’ agreement between manual review by a pathologist in a reference laboratory and automated review using image analysis. Table 3 also reveals that in this study image analysis reported a lower number of equivocal cases than the manual pathology review. Indeed, of the 17 cases re-classified by image analysis, 15 had been FISH tested and in each of these cases the gene amplification status was concordant with the reclassified score by image analysis, suggesting that image analysis would have led to a significant cost saving in this instance. FIG. 2 shows representative images from the system and illustrates the ability of the HER-2 algorithm to detect regions of positively and continuously stained cell membrane.

TABLE 3
Performance of Image Analysis with clinical samples assessed
on the basis of the ASCO/CAP and UK scoring guidelines.

| | | Image Analysis Classification | | | |
|---|---|---|---|---|---|
| | | 0/1+ | 2+ | 3+ | Total |
| Manual | 0/1+ | 178 | 5 | 0 | 183 |
| Classification | 2+ | 15 | 23 | 2 | 40 |
| | 3+ | 0 | 3 | 49 | 52 |
| Total | | 193 | 31 | 51 | 275 |

Concordance: 90.9%
Kappa: 0.811

As expected, analysis of the corresponding cell-line control material determined that those slides stained using automated systems exhibited less variance than those prepared manually. Nonetheless, normalisation to compensate for variance had no impact on the classification of HER-2 by image analysis.

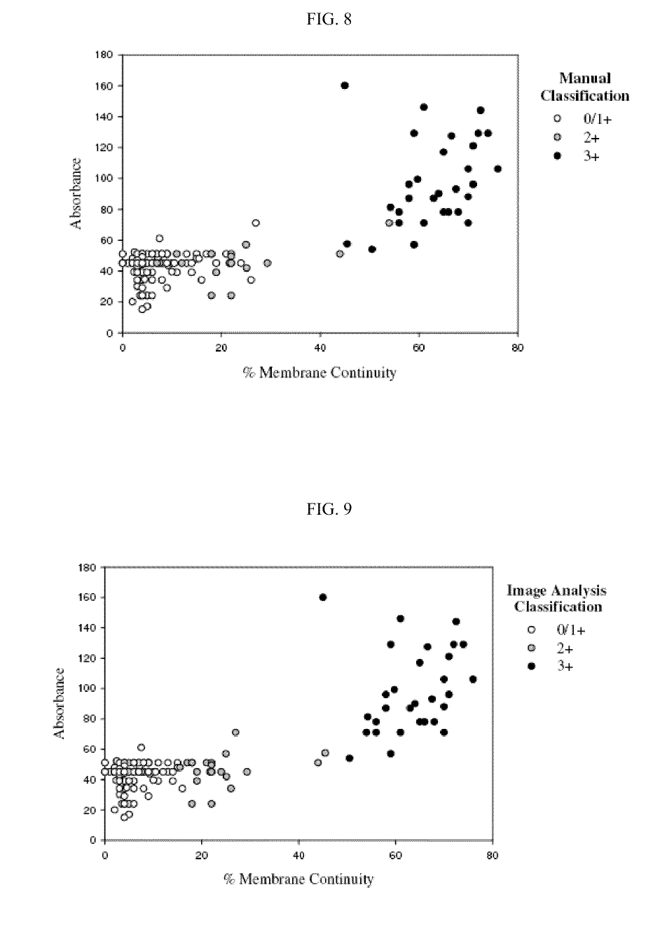

FIGS. 8 and 9 show how the membrane continuity and absorbance values determined according to the present method translate into a HER-2 score via manual classification and image analysis respectively. It is apparent from these figures that there is a very poor distinction of 0/1+ and 2+ cases on basis of membrane intensity (absorbance) alone. However the combination of membrane continuity and absorbance values is highly predictive of HER-2 score, and enables a much clearer separation of HER-2 categories. There is close concordance between the manual classification and image analysis according to the present method.

Concordance with FISH Evaluation

A number of cases in the study (136) were also analysed by FISH, the ‘gold-standard’ method of HER-2 evaluation. The concordance rate between HER-2 gene amplification and IHC review was determined to be excellent for both image analysis and the pathology review, demonstrating that image analysis can robustly and accurately classify HER-2 status (Table 4). However, it was observed that image analysis review of the IHC sections attained a slightly higher concordance rate with FISH than the manual review (95% versus 92% respectively). Although both methods correctly classified 13 FISH positive cases as 3+ IHC cases, quantitation by image analysis identified 92 cases with no gene amplification, in comparison with 83 for the pathology review. This was attributed to improved differentiation between negative and equivocal cases by image analysis and suggests that the automated method of review is more accurate than visual scoring.

TABLE 4
Concordance between HER-2 gene amplification and HER-2 protein
expression reviewed by a pathologist and by image analysis.

| | IHC | | | | | |
|---|---|---|---|---|---|---|
| | Manual Classification | | | Image Analysis Classification | | |
| | Negative | Positive | Equivocal | Negative | Positive | Equivocal |
| FISH | (0/1+) | (3+) | (2+) | (0/1+) | (3+) | (2+) |
| Positive | 7 | 13 | 7 | 6 | 13 | 8 |
| Negative | 83 | 1 | 25 | 92 | 0 | 17 |
| Concordance | 92% | 93% | n/a | 94% | 100% | n/a |

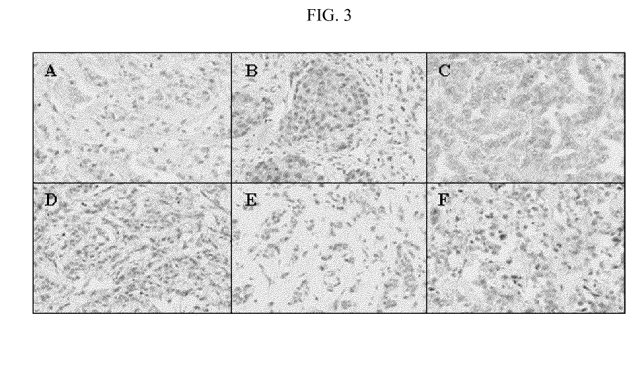

Nonetheless, it is evident from Table 4 that significant disagreement between IHC classification and FISH amplification occurred in 6 cases which were subsequently re-examined for possible causes of conflicting results. FIG. 3 illustrates that IHC staining was negligible in all of these cases and the pathology and image analysis reviews agreed that these should be categorised as negative or 0/1+. However, in each case gene amplification was determined to be positive by FISH, suggesting these are false-negative IHC cases. A number of previous studies have reported this phenomenon in approximately 7% of HER-2 FISH positive results which would correlate with the figures determined here, and the cause is generally attributed to destruction of the HER-2 epitope or antigen loss during fixation or processing. It was noted that if these slides were omitted from Table 4 the sensitivity of both review methods would be significantly improved, with HER-2 classification by image analysis review achieving 100% sensitivity and specificity.

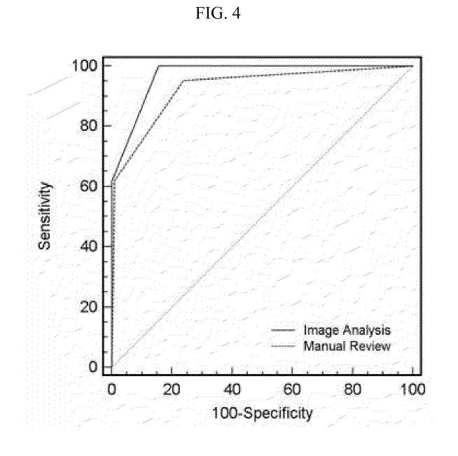

Receiver-operating characteristic (ROC) curve analysis was used to compare the accuracy of the manual and image analysis methods with FISH evaluation as the standard (FIG. 4). The area under the curve (AUC) value was found to be 0.93 (95% CI 0.867-0.965) for the manual review with 0.97 (95% CI 0.925-0.992) obtained by image analysis, confirming the accuracy of the automated algorithm. Both review methods were found to be statistically significant (P<0.0001).

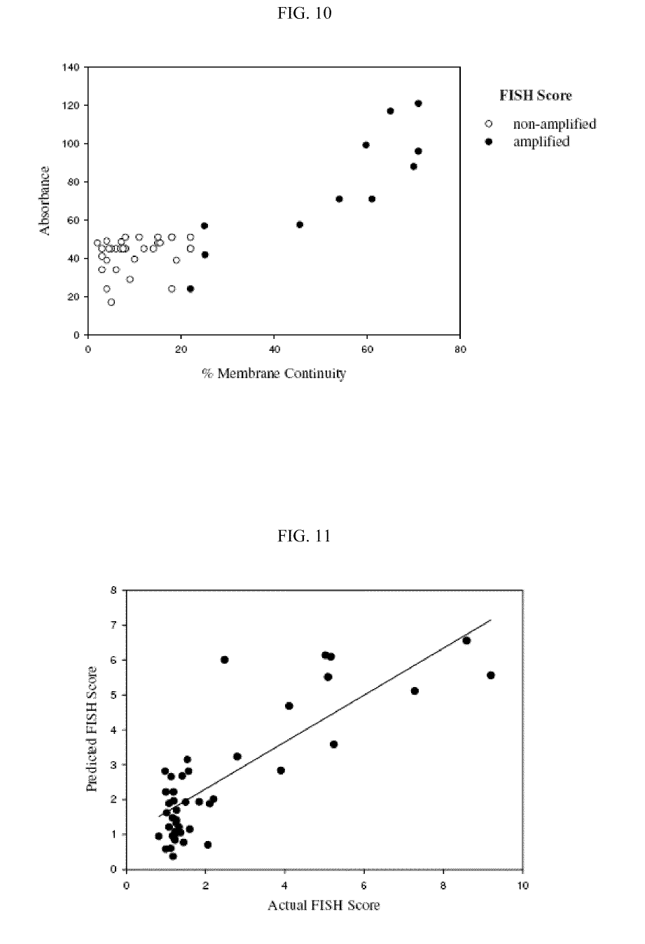

FIGS. 10 and 11 show how membrane continuity together with membrane absorbance is highly predictive of FISH score, i.e. can be used to distinguish between amplified and non-amplified cases.

Comparison with Other Commercially Available Image Analysis Systems

A number of other image analysis systems are commercially available for use as a decision support tool in the clinical setting. Nevertheless, Table 5 illustrates that the accuracy in predicting HER-2 status varies considerably across the offerings and a number of key distinguishing factors exist between the systems. In comparison to the data submitted by other systems for FDA approval, this validation study across a larger cohort of clinical cases has established that Tissue IA achieved a 5-14% higher correlation with manual review. Indeed, the 91% concordance rate reported here is substantially greater than the 70% agreement detailed by Camp et al., 2003, using the AQUA system. In addition, the performance of the HER-2 algorithm under trial has attained high levels of concordance with gene amplification status, greater than that reported for the ACIS system by Tawfik et al., 2006, and Wang et al., 2001. Furthermore, this system has been validated to perform with slides stained using Dako HercepTest®, Leica Oracle™ and Ventana Pathway® HER-2 antibodies.

TABLE 5
Comparison of SlidePath's Tissue IA system with other
commercially available systems for HER-2 analysis.

| | | | | Dako | Ventana | |
|---|---|---|---|---|---|---|
| Manufacturer | SlidePath | Aperio | BioImagene | (Chromavision) | (TriPath Imaging) | |
| System | Tissue IA | Scanscope | Pathiam | ACIS | VIAS | |
| | | XT | | | | |
| Assay | Dako | Dako | Dako | Dako | Ventana | Ventana |
| | HercepTest | HercepTest | HercepTest | HercepTest | Pathway | Pathway |
| | Leica Oracle | | | | (4b5) | (cb11) |
| | Bond | | | | | |
| | Ventana | | | | | |
| | Pathway (4b5) | | | | | |
| Concordance | 91% | 86% * | 81% * | 75% * | 86% * | 77% * |
| with Manual | (n = 275) | (n = 180) | (n = 176) | (n = 90) | (n = 206) | (n = 201) |
| Review | | | | | | |
| (Sample size) | | | | | | |

| | | | | | |
|---|---|---|---|---|---|
| Image Format | | ◯ | [symbol] | ◯ | ◯ |
| Support | | | | | |
| Dependence | | | | | |
| on manual | | | | | |
| selection | | | | | |
| Quantitation | Intensity, | Intensity | Morphology, | Intensity | Intensity |
| Base | Continuity | | Intensity | | |

High , Intermediate [symbol] , Low ◯
* Data from FDA 510k substantial equivalence reports (www.fda.gov)

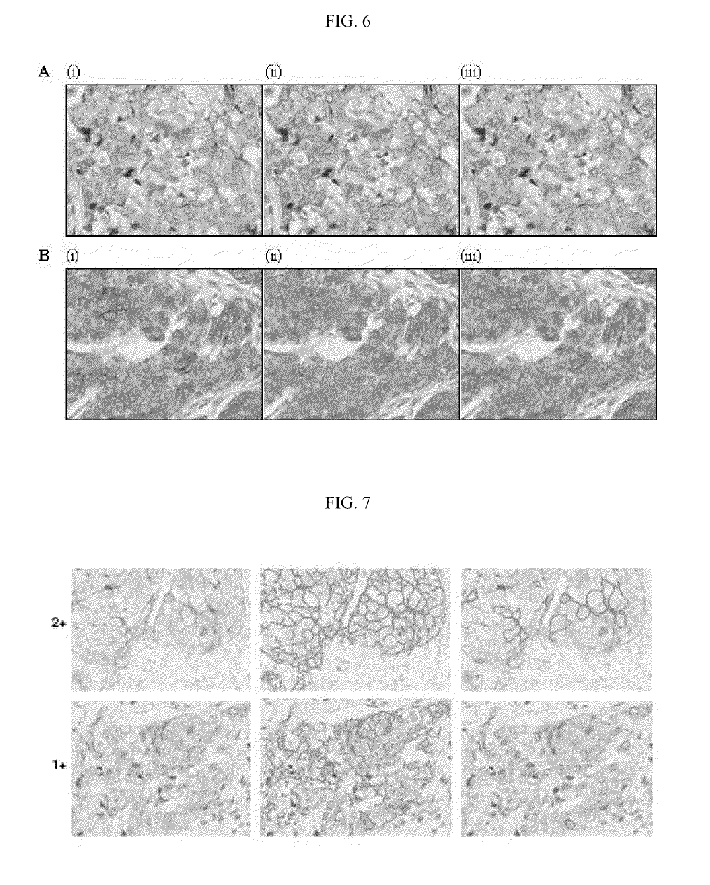

The disparity in the accuracy of the image analysis systems may be attributed to a variety of factors. However, it is evident from Table 5 that the distinguishing factor between the image analysis systems is the quantitation base used to determine the extent of HER-2 protein expression. Whilst all algorithms quantify the intensity of membrane staining the algorithm under trial also determines the continuity of the membrane staining, the parameter which underpins the definition of positive HER-2 status. Although staining intensity is critical for distinguishing the 3+ cases, consideration of membrane continuity is essential for clear distinction of the 0/1+ and the equivocal 2+ categories. Indeed, FIG. 6 demonstrates that although the intensity of membrane staining can appear to be similar for both groups, the extent of continuity of that staining is undoubtedly a distinguishing factor which enables correct differentiation of a number of ambiguous visual IHC scores. FIG. 7 shows further examples of how 1+ and 2+ cases can be discriminated by considering the continuity of membrane staining.

Discussion

High levels of HER-2 protein expression or HER-2 gene amplification are used to identify patients for whom trastuzumab may be of benefit for treatment of breast cancer in the metastatic or adjuvant disease settings. In accordance with the HER-2 testing guidelines, in most laboratories IHC is carried out first with additional testing accomplished by FISH. However, assignment of HER-2 grade by assessment of IHC is inherently subjective and dependent on the skill and experience of the reviewing pathologist. Thus the standardisation of diagnosing breast cancer is a very important task for improving personalised cancer patient care as a cancer patient to whom an inappropriate drug is given will face disease progression during the treatment time impacting on overall survival rate and increased costs.

The last 10 years have seen enormous advances in the capabilities of image analysis systems applied to tissue sections with complex computer algorithms used to interpret the images. Digital microscopy is increasingly being used to document and analyse tissue specimens in modern research laboratories and it has recently been proposed that newly introduced image analysis technology has a major role to play in the progress of diagnostic pathology. In comparison with human-based assessment, automated image analysis offers numerous advantages such as precise, reproducible, continuous and objective assessment of protein expression. Indeed, image analysis has been used to evaluate the expression of nuclear markers such as oestrogen and progesterone receptor; cytoplasmic markers such as B-catenin; and other membrane proteins such as E-cadherin. Nonetheless, a major requisite for the acceptance of image analysis in the clinical laboratory is that it must yield high concordance with the current gold standard method. Indeed, although the ASCO/CAP guidelines have advocated the use of image analysis for HER-2, a degree of resistance to its adoption in the clinical setting has been observed, perhaps due to the low accuracy and restrictions of the currently available and approved systems.

The present invention has aimed to address the inherent deficiencies in other systems. In the first instance, the algorithm used in embodiments of the present invention measures the continuity of membrane staining as well as the staining intensity, and has demonstrated that consideration of both parameters enables accurate distinction of HER-2 status. Furthermore, this HER-2 algorithm has been validated to perform with some of the most prevalent HER-2 antibodies on the market. Although the HER-2 guidelines for testing do not stipulate the use of a particular antibody, the Dako HercepTest® and Ventana Pathway® are recommended as FDA approved kits, and the Leica Oracle™ HER-2 antibody is also frequently employed in laboratories who have demonstrated concordance with a validated method.

Our findings demonstrate that image analysis can accurately and robustly classify HER-2 status. A concordance rate of 91% was observed in comparison with manual review by a pathologist, and the significant value of image analysis was exemplified by a 4% reduction in the reporting of equivocal cases. This represents a decrease in the number of cases requiring confirmatory FISH testing and thus a potential cost saving for clinical laboratories. Moreover, the concordance of image analysis with gene amplification status as the standard was observed to be 95% which represents better correlation and accuracy with FISH than the manual interpretation of IHC. The data from this study is substantially greater than that reported by existing systems in FDA approval documentation and independently by Camp et al., 2003, using the AQUA platform, or Wang et al., 2004, and Tawfik et al., 2006, using the ACIS system. Indeed, in comparison with FISH, the ACIS system was demonstrated to falsely predict 4-11% of cases as HER-2 amplified, which could have a significant impact on patient welfare. In contrast, the method of the present invention accurately predicted all HER-2 gene amplified cases with a false positive rate of 0%.

Although it is generally accepted that the standard assessment of IHC will remain the manual pathology review, our findings suggest that integration of image analysis into the diagnostic workflow would significantly enhance the reproducibility of scoring, particularly in those laboratories where there is a lack of experience in interpreting HER-2 staining However, aside from providing assistance for interpretation, image analysis could be utilised as an internal resource to qualify the quality of IHC staining, introducing an unprecedented level of internal laboratory quality assurance.

The recent reports of poor observer variability regarding the evaluation of HER-2 in the clinical setting justify the development of software tools to help standardise interpretation, particularly in equivocal cases. Based on this study, the method of the present invention has been validated as a consistent scoring tool with excellent levels of concordance with manual scoring and FISH, advocating the use of the method as a decision support system for pathologists to assist in the diagnosis of disease.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments and that many modifications and additions thereto may be made within the scope of the invention. Indeed, various modifications of the described modes for carrying out the invention which are obvious to those skilled in the art are intended to be within the scope of the claims. Furthermore, various combinations of the features of the following dependent claims can be made with the features of the independent claims without departing from the scope of the present invention.

1. A method for detecting an expression level of a cell surface marker in a sample, comprising:
(a) staining the sample with a reagent that labels the cell surface marker;
(b) obtaining an image of the stained sample; and
(c) determining a value for continuity of cell surface staining in the image,
wherein the value is indicative of the expression level.

2. A method according to claim 1, wherein the cell surface marker is a membrane protein.

3. A method according to claim 1, wherein the cell surface marker is a growth factor receptor.

4. A method according to claim 1, wherein the cell surface marker is associated with cancer.

5. A method according to claim 1, wherein the cell surface marker is HER-2.

6. A method according to claim 1, wherein step (a) comprises staining the sample by immunohistochemistry using an antibody specific for the cell surface marker.

7. A method according to claim 1, wherein steps (b) and (c) are performed by an automated image capture and analysis system.

8. A method for automated analysis of an image of a stained tissue sample, comprising determining a value for continuity of cell surface staining in the image.

9. A method according to claim 8, wherein the sample has been stained with a reagent that labels a cell surface marker, and the value is indicative of an expression level of the cell surface marker in the sample.

10. A method according to claim 8, wherein pixels in the image representative of positive staining are detected by applying a colour transformation to the pixels, and applying a threshold value to suppress background.

11. A method according to claim 8, wherein pixels in the image representative of cell surfaces are determined by detecting pixels surrounding nuclei stained with a counterstain.

12. A method according to claim 8, wherein the continuity value comprises a percentage of cell surfaces in the image which are continuously stained.

13. A method according to claim 8, further comprising determining a value for intensity of cell surface staining in the image.

14. A method according to claim 13, wherein the continuity value and intensity value are combined to provide a weighted probability value indicative of a probability of the sample being classified in a predefined staining class.

15. A method according to claim 13, wherein the sample is classified into a staining class indicative of a level of HER-2 expression in the sample.

16. A method for diagnosing a condition associated with expression of a cell surface marker in a subject, comprising detecting an expression level of the cell surface marker in a sample from the subject by a method as defined in claim 1, wherein an elevated expression level of the cell surface marker in the sample compared to a control sample is indicative of the presence of the condition in the subject.

17. A method according to claim 16, wherein the condition is cancer.

18. A method according to claim 17, wherein the cell surface marker is HER-2.

19. A method for predicting responsiveness to therapy with an anti-HER-2 antibody in a subject, comprising detecting an expression level of HER-2 in a sample from the subject by a method as defined in claim 5, wherein an elevated expression level of HER-2 in the sample compared to a control sample is indicative of responsiveness of the subject to therapy with the anti-HER-2 antibody.

20. A method according to claim 19, wherein the expression level of HER-2 is classified as a score of 0, 1+, 2+ or 3+, and a score of 3+ or above is indicative of responsiveness of the subject to therapy with the anti-HER-2 antibody.

21. A computer program, residing on a computer-readable medium, for automated image analysis, comprising machine-readable instructions for performing a method comprising determining a value for continuity of cell surface staining in an image of a stained tissue sample.

22. An automated imaging apparatus, wherein the apparatus is configured to obtain an image of a stained tissue sample, and determine a value for continuity of cell surface staining in the image, wherein the value is indicative of an expression level of a cell surface marker in the sample.
